(19) United States
(12) Patent Application Publication
(10) Pub. No.: US 2011/0288042 A1
(43) Pub. Date: Nov. 24, 2011

(54) STABLE HIGHLY PURE AZACITIDINE AND PREPARATION METHODS THEREFOR

(75) Inventors: Alex Weisman, Kiriat Ekron (IL); Lior Zelikovitch, Mazkeret Batya (IL); Oded Friedman, Talmey Yechiel (IL); Josef Manascu, Omer (IL)

(73) Assignee: CHEMAGIS LTD., Bnei Brak (IL)

(21) Appl. No.: 12/671,663

(22) PCT Filed: Jul. 23, 2008
(86) PCT No.: PCT/IL08/01015

§ 371 Date: Aug. 9, 2011

Related U.S. Application Data
(60) Provisional application No. 60/963,113, filed on Aug. 2, 2007.

(51) Int. Cl. A61K 31/706; C07H 19/12; C07H 1/06

(52) U.S. Cl. 514/43; 536/28.3

(57) ABSTRACT

Disclosed herein are methods of obtaining highly pure 5-azacytidine, which contains minimal amounts of degradation impurities and methods of assessing the impurity profile of the degradation of cytidine analogues, such as 5-azacytidine

### CROSS-REFERENCE TO RELATED APPLICATIONS

This patent application claims the benefit of U.S. Provisional Patent Application No. 60/963,113, filed Aug. 2, 2007, which is incorporated herein by reference.

### TECHNICAL FIELD

The present invention relates to methods of obtaining highly pure azacitidine containing minimal quantities of degradation impurities.

### BACKGROUND OF THE INVENTION

Azacitidine, (5-azacytidine, Compound I), marketed by Pharmion under the trademark VIDAZA™, is the first drug approved by the United States Food and Drug Administration (FDA) for treating myelodysplastic syndromes (MDS), a diverse collection of hematological conditions united by ineffective production of blood cells and varying risks of transforming into acute myelogenous leukemia. Azacitidine is an anticancer medicine that exerts its antineoplastic effect by causing hypomethylation of DNA and direct cytotoxicity on abnormal hematopoietic cells in the bone marrow and thus is used for treating certain types of bone marrow cancers and blood cell disorders.

Azacitidine is an azacytosine nucleoside, having the chemical name 4-amino-1-β-D-ribofuranosyl-1,3,5-s-triazine-2(1H)-one, and the chemical structure:

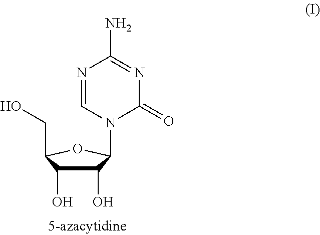

Azacitidine is a white to off-white solid, which is insoluble in acetone, ethanol, and methyl ethyl ketone; slightly soluble in ethanol/water (50/50), propylene glycol, and polyethylene glycol; sparingly soluble in water, water saturated octanol, 5% dextrose in water, N-methyl-2-pyrrolidone, normal saline, and 5% Tween 80 in water; and soluble in dimethylsulfoxide (DMSO).

VIDAZA™ may be administered subcutaneously, wherein the drug is supplied in the form of a sterile powder for reconstitution and subcutaneous injection in vials containing 100 mg of azacitidine and 100 mg of mannitol as a lyophilized powder. Another route of administration is through a slow intravenous infusion over a period of 10-40 minutes.

5-Azacytidine first was prepared via a multi-step synthesis starting from peracetylated 1-glycosyl isocyanate by Piskala and Sorm (Collect. Czech. Chem. Commun., 29, 2060, 1964). Subsequently, 5-azacytidine was isolated as a new antibiotic by Hanka, et al. (Antimicrob. Ag. Chemother., 619, 1966) from Streptoverticillium ladakanus.

U.S. Pat. No. 7,038,038 (hereinafter the '038 patent) describes a process for preparing 5-azacytidine, which comprises the steps of: (a) reacting 5-azacytosine with a silylating reagent, e.g., 1,1,1,3,3,3-hexamethyldisilazane (HMDS), in the presence of ammonium sulfate at elevated temperature to yield a silylated 5-azacytosine, (b) coupling the reaction mixture of step (a) with 1,2,3,5-tetra-O-acetyl-β-D-ribofuranose in dichloromethane in the presence of TMS-triflate followed by treatment with a mixture of sodium carbonate and sodium bicarbonate, (c) deprotecting the silylated azacitidine product of step (b) by adding sodium methoxide in methanol, and (d) purifying crude 5-azacytidine by crystallization from mixture of DMSO and methanol. The '038 patent does not disclose the purity of the obtained 5-azaytidine.

The following Scheme 1 illustrates the process of the '038 patent:

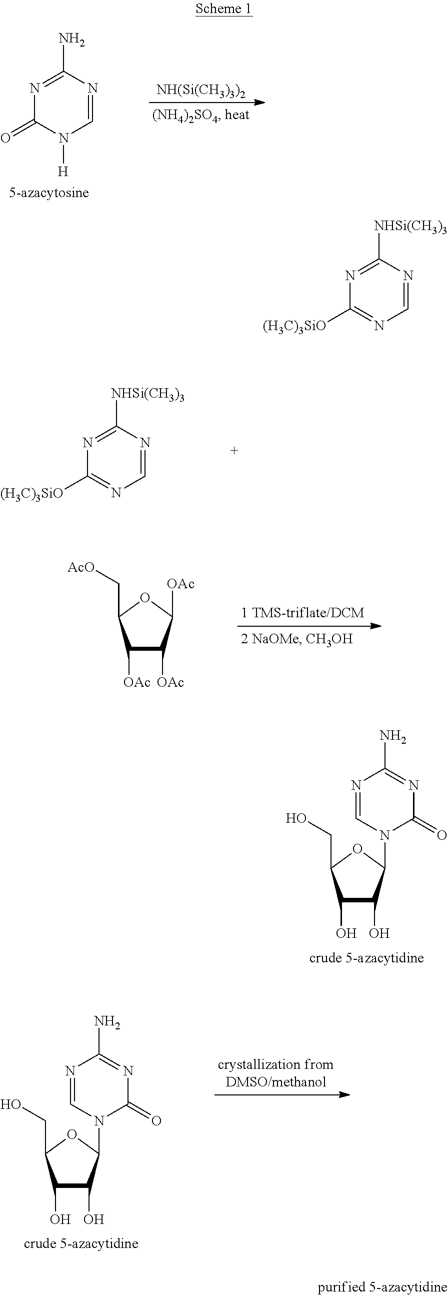

A different procedure for preparing 5-azacytidine, which is based on the procedure of Vorbrueggen et. al., J. Org. Chem. Vol. 39, No.25, 1974, is described in Scheme 2 below. The process comprises the steps of: (a) reacting 5-azacytosine with a silylating reagent, e.g., 1,1,1,3,3,3-hexamethyldisilazane (HMDS), in the presence of ammonium sulfate at elevated temperature to yield a silylated 5-azacytosine, (b) coupling the reaction mixture of step (a) with 1,2,3,5-tetra-O-acetyl-β-D-ribofuranose in acetonitrile in the presence of stannic chloride (SnCl₄), and (c) deprotecting the silylated azacitidine product of step (b) by adding sodium methoxide in methanol.

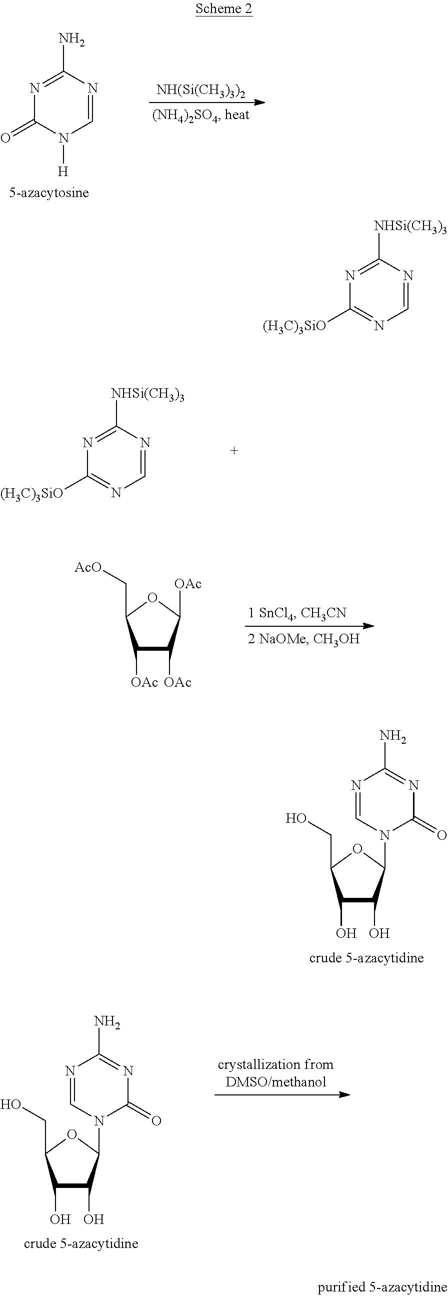

U.S. Pat. No. 6,887,855, U.S. Pat. No. 6,943,249 (hereinafter the '249 patent), and U.S. Pat. No. 7,078,518 (hereinafter the '518 patent) describe eight crystalline forms of 5-azacytidine designated as forms I-VIII, along with an amorphous form. According to the examples of the '249 patent, Form I of 5-azacytidine is obtained by crystallization from solvent mixtures comprising a primary solvent (DMSO) and a co-solvent (e.g., ethanol, isopropanol, acetonitrile, etc.), but the '249 patent is silent with regard to the purity of the obtained product. It is mentioned in Example 1 of the '518 patent that the crude azacitidine was dissolved in DMSO preheated to about 90° C., then methanol was added to the DMSO solution. The co-solvent mixture was cooled to allow crystallization of 5-azacytidine crystals and the product was collected by filtration and dried. According to Examples 2, 3, and 4 of the '518 patent, 5-azacytidine was re-crystallized from solvent mixtures of DMSO/toluene, DMSO/methanol, and DMSO/chloroform, and from N-methyl-2-pyrrolidone as a single solvent, but in no example was the purity or yield of the obtained product reported.

R. E. Notari and J. L. DeYoung in Pharmaceutical Science, Vol. 64, No. 7, July 1975, p 1148-1157, investigated the stability of 5-azacytidine in aqueous solution, concluding that it was relatively instable in comparison to cytidine. The hydrolytic degradation of 5-azacytidine was studied as a function of pH, temperature, and buffer concentration. For example, at pH 1, the main degradation products were 5-azacytosine and 5-azauracil, while at higher pH values, the degradation products were different. However, these degradation products were not detectable while being examined in acidic solutions as they were non-chromophoric. The following Scheme 3 describes the degradation products:

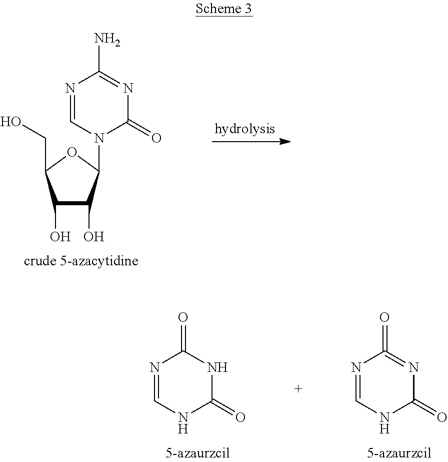

In another study, conducted by J. A. Beisler, Journal of Medicinal Chemistry, Vol. 21, No. 27, 1978, p 204-208, it is mentioned that during the prolonged intravenous infusion time of 5-azacytidine, facile drug decomposition occurs in aqueous formulations giving rise to products of unknown toxicity. Thus, HPLC analysis of 24 hours old aqueous solutions of 5-azacitidine revealed that the main degradation products are N-(formylamidino)-N′-β-D-ribofuranosylurea (Compound IV, RGU-CHO) and 1-β-D-ribofuranosyl-3-guanylurea (Compound V, RGU). The following Scheme 4 depicts the degradation products:

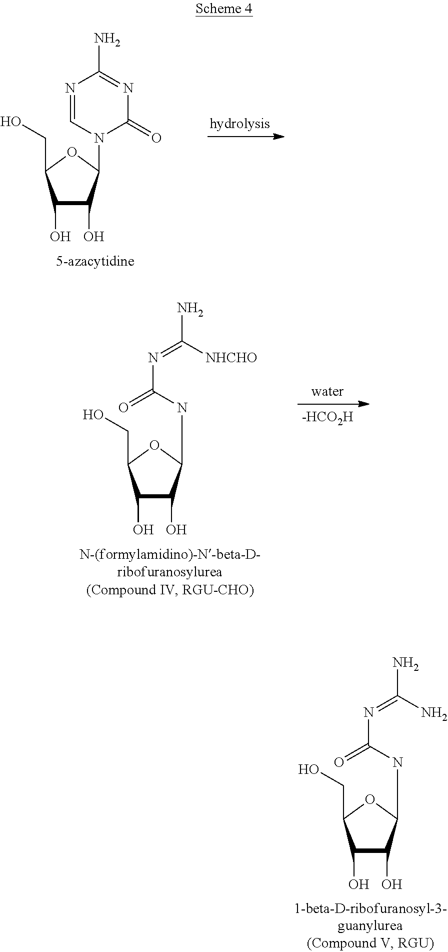

Thus, it is evident that 5-azacytidine is not stable and is prone to degradation in aqueous formulations. Furthermore, it is likely that purification of 5-azacytidine from a solvent that contains water will be not effective, due to a high level of instability in the presence of water. Hence, it is likely to find relatively high levels of degradation products in the commercial product. Therefore, there is a need for improved methods of preparing highly pure 5-azacytidine, which contains minimal amounts of degradation products, such as N-(formylamidino)-N′-β-D-ribofuranosylurea, particularly on a commercial scale. The present invention provides such methods, as will be apparent from the description of the invention provided herein.

### SUMMARY OF THE INVENTION

It has been found by the inventors of the present invention, that while analyzing a sample of the drug VIDAZA™, which was purchased as a ready-to-use dosage form for pharmaceutical use, the purity of the compound 5-azacytidine was only 98.45%. Furthermore, the sample analysis showed that significant quantities of impurities were contained in the sample, which were identified as degradation products of 5-azacytidine.

Thus, the present invention provides methods of preparing highly pure 5-azacytidine, i.e., containing minimal amounts of degradation products, which is suitable for prolonged intravenous infusions, comprising:
(a) heating a solution of crude 5-azacytidine to at least about 45° C.;
(b) allowing the solution of step (a) to cool to precipitate crystals of purified 5-azacytidine from the solution;
(c) optionally isolating, washing, and drying the crystals of step (b); and
(d) optionally slurrying the crystals of step (c) in a solvent, and filtering and drying the filtered crystals. In some embodiments, the isolating of step (c) comprises filtering.

In some cases, 5-azacytidine obtained by the methods provided herein, has a purity of at least 99% by weight, or at least 99.6% by weight.

In various cases, 5-azacytidine obtained by the methods provided herein contains less than about 0.2% by weight of at least one degradation product. In specific cases, the 5-azacytidine contains less than about 0.2% by weight N-(formylamidino)-N′-β-D-ribofuranosylurea (Compound IV, RGU-CHO) and/or less than about 0.1% of 1-β-D-ribofuranosyl-3-guanylurea (Compound V, RGU).

The present invention also provides a method of analyzing the impurity profile of 5-azacytidine, typically using chromatography, such as liquid or gas chromatography. Methods of liquid chromatography include, for example, Thin Layer Chromatography (TLC), High Pressure Liquid Chromatography (HPLC), and/or Liquid Chromatography/Mass spectrometry (LC-MS).

The method of analyzing the impurity profile of azacitidine typically comprises:
separating a sample comprising 5-azacytidine in an eluent using a liquid chromatography system (LC), wherein the LC system is equipped with a suitable stationary phase and is capable of separating the 5-azacytidine and any degradation products present in the sample; and
identifying, detecting, or both the presence of any degradation products in the sample using mass spectrometry (MS).

A sample of 5-azacytidine, which was withdrawn from the VIDAZA™ packaging for injectable suspension, was analyzed using the HPLC method detailed in Example 8, below. Three impurities were identified: RGU (Compound V), RGU-CHO (Compound IV) and Compound VI.

The present invention further provides a method of analyzing the degradation products of cytidine analogues, such as 5-azacytidine, 5-aza-2′-deoxycytidine, and zebularine (which is reported as stable in aqueous solution), that can be useful to establish a degradation pathway of the cytidine analogue, 5-azacytidine, when exposed to degradation-inducing conditions.

According to one embodiment of the present invention, an induced degradation study on 5-azacytidine can be carried out in solid state conditions, as well as in liquid state conditions. Solid state conditions that can be used include, but are not limited to, storage conditions, ambient conditions, elevated temperature conditions, UV light conditions, and accelerated conditions. The liquid state conditions that can be used include, but are not limited to, photolysis conditions, acidic conditions, basic conditions, and oxidative conditions.

### BRIEF DESCRIPTION OF THE FIGURES

FIG. 1 depicts the thermogravimetric analysis (TGA) curve of the 5-azacytidine obtained according to Reference Example 1A

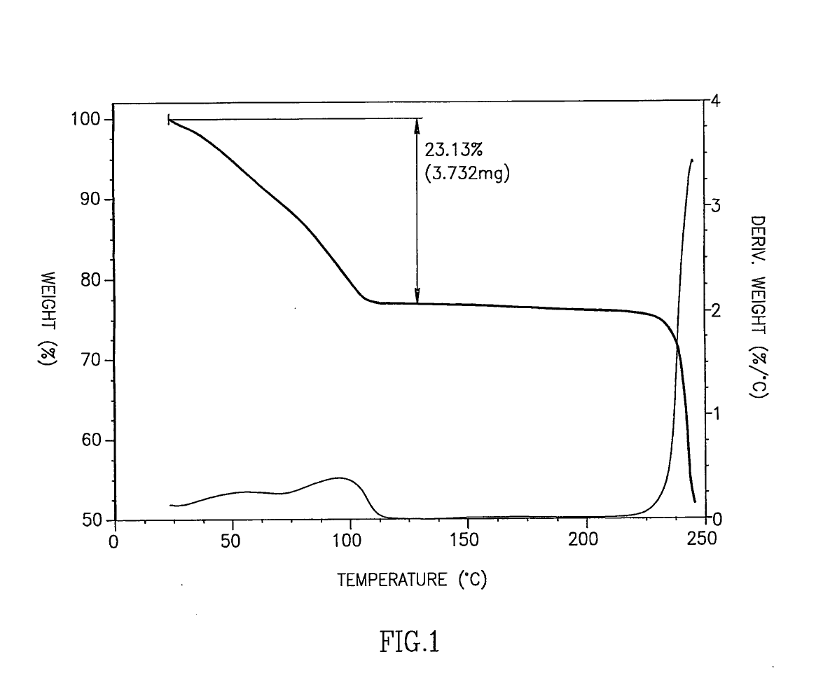

FIG. 2 depicts the thermogravimetric analysis (TGA) curve of the 5-azacytidine obtained according to Reference Example 1B, entry 1.

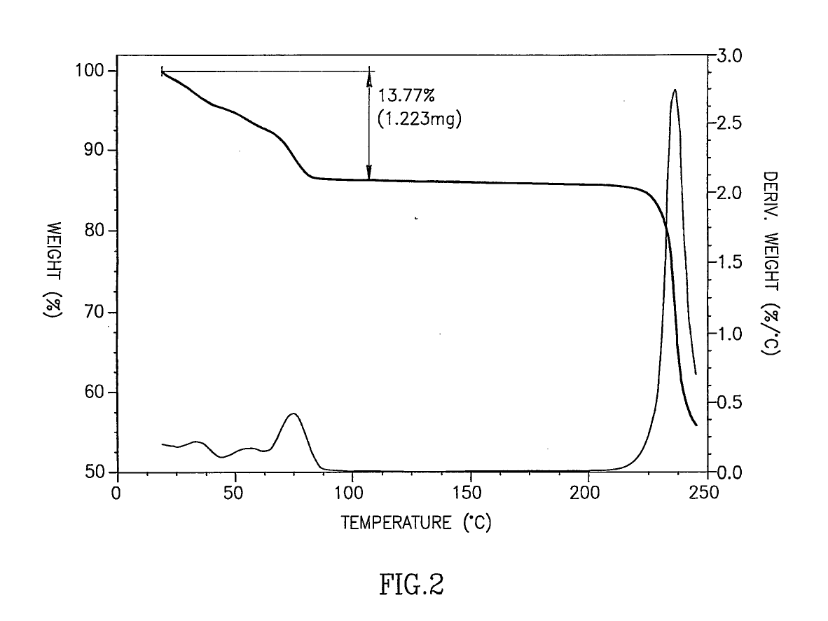

FIG. 3 depicts the thermogravimetric analysis (TGA) curve of the 5-azacytidine obtained according to Reference Example 1B, entry 2.

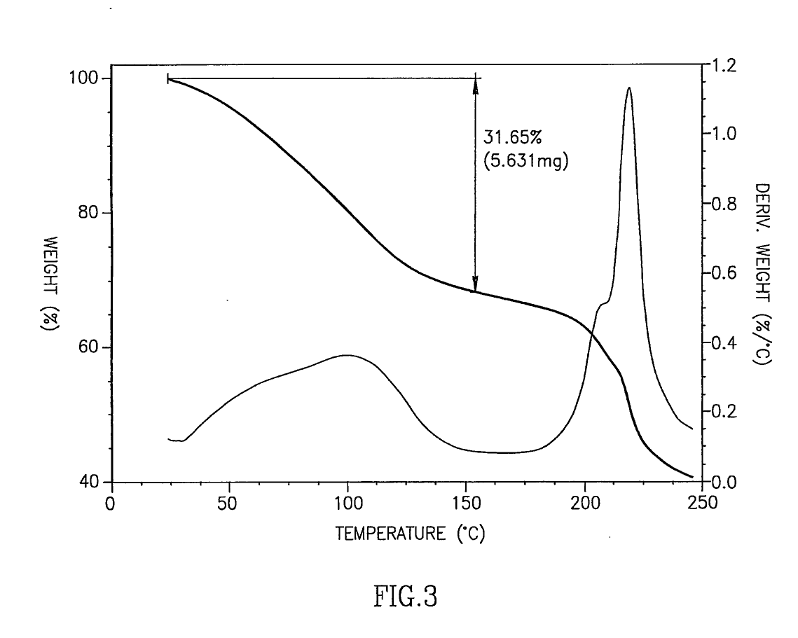

FIG. 4 depicts the thermogravimetric analysis (TGA) curve of the 5-azacytidine obtained according to Reference Example 1B, entry 3.

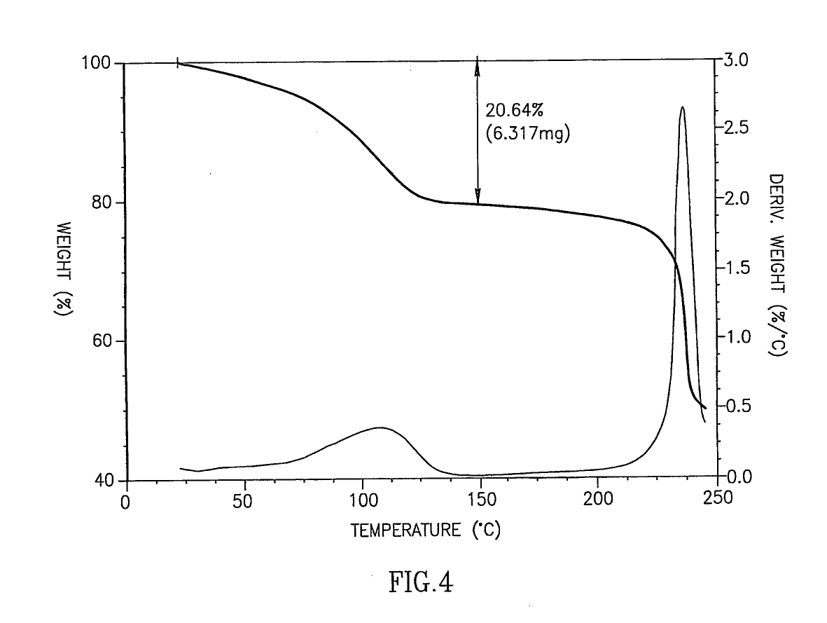

### DETAILED DESCRIPTION OF THE INVENTION

In one embodiment, the present invention provides methods of preparing pure 5-azacytidine, containing less than 0.2% by weight of at least one degradation product, which can be used for prolonged intravenous infusions, comprising:
(a) heating a solution of crude 5-azacytidine to at least about 45° C.;
(b) allowing the solution of step (a) to cool to precipitate crystals of purified 5-azacytidine from the solution;
(c) optionally isolating, washing, and drying the crystals of step (b); and
(d) optionally slurrying the crystals of step (c) in a solvent, and filtering and drying the filtered crystals.

As used herein, the term “crude 5-azacytidine” refers to a 5-azacytidine sample having a purity up to 98.9% by weight, preferably up to about 98.5% by weight of 5-azacytidine. As used herein, the term “pure 5-azacytidine” or “purified 5-azacytidine” refers to a 5-azacytidine having a purity of at least 99.0% by weight, preferably at least 99.5% or at least 99.6% by weight of 5-azacytidine.

The solutions of crude 5-azacytidine can be heated to a temperature of at least about 45° C. The temperature can be at least about 50° C., at least about 55° C., at least about 60° C., at least about 65° C., at least about 70° C., at least about 75° C., at least about 80° C., at least about 85° C., at least about 90° C., at least about 95° C., or at least about 100° C. The temperature to which the solution is heated depends upon the solvent used to prepare the solution and the solvent's physical properties (e.g., boiling point), a determination of which is within the skill of a person of the relevant art.

Preferably, the solution of the crude 5-azacytidine is prepared using an organic solvent, non-limiting examples of which are N,N-dimethylformamide (DMF), N,N-dimethylacetamide (DMA), ethylene glycol, N-methyl-2-pyrrolidone, dimethylsulfoxide (DMSO), and mixtures thereof. In more preferred embodiments, the solvent is N,N-dimethylformamide (DMF), N,N-dimethylacetamide (DMA), or mixtures thereof.

Preferably, the solvents used for slurrying the crystals of 5-azacytidine include, but are not limited to, acetone, methyl ethyl ketone, methyl isobutyl ketone, ethyl acetate, n-propyl acetate, isoproyl acetate, n-butyl acetate, isobutyl acetate, ethanol, and mixtures thereof.

Preferably, the ratio of the crude 5-azacytidine to the solvent used in step (a), i.e., 5-azacytidine: solvent ratio, is about 1 gram (g) 5-azacytidine per at least 2 milliliter (ml) of solvent, preferably the ratio is about 1 g 5-azacytidine per about 10 to about 20 ml of solvent.

Preferably, 5-azacyitidine obtained by methods provided herein has a purity of at least 99% by weight, or at least 99.6% by weight. Preferably, 5-azacytidine obtained by methods provided herein contain less than about 0.2% by weight of N-(formylamidino)-N′-β-D-ribofuranosylurea (Compound IV, RGU-CHO) and/or less than about 0.1% by weight of 1-β-D-ribofuranosyl-3-guanylurea (Compound V, RGU).

According to the guidance “Q3C: Residual Solvents” published by the “International Conference on Harmonization of Technical Requirements of Registration of Pharmaceuticals for Human Use (ICH)” [A copy of this guidance can be found in the US Federal Register Volume 62, No. 247 (Dec. 24, 1974) Docket 97D-0148, Appendixes 5-7: toxicological data for class 1-3 solvents respectively], the use of industrial solvents in active pharmaceutical ingredients is restricted according to their toxicity and safety features. The industrial solvents are divided into three main classes:

Class 1: Solvents to be avoided. These are solvents that should not be employed in the manufacture of drug substances or drug products because of their unacceptable toxicity or their deleterious environmental effect. Solvents that belong to this class are: benzene, carbon tetrachloride, 1,2-dichloroethane and others.

Class 2: Solvents to be monitored. These are solvents that should be limited in pharmaceutical products because of their inherent toxicity. Important industrial solvents that belong to this class are chlorinated solvents such as chloroform, dichloromethane, hydrocarbons such as hexane and aromatic solvents such as toluene.

Class 3: Solvents that are regarded as less toxic and of lower risk to human health. Important industrial solvents that belong to this class are certain ketones, esters, alcohols and others.

For example, according to the above mentioned Q3C guidance, the maximal concentration limit of some relevant solvents is summarized in Table 1.

TABLE 1

| Solvent | Class | Maximal permitted concentration, ppm |
|---|---|---|
| Chloroform | 2 | 60 |
| Methanol | 2 | 3000 |
| Toluene | 2 | 890 |
| DMSO | 3 | 5000 * |
| DMF | 2 | 880 |
| Acetone | 3 | 5000 * |

* The permitted level of a class 3 solvent is 5000 ppm (0.5%).

It has been found by the inventors of the present invention that the purification of 5-azacytidine by crystallization according to Example 2 or 3 of Patent U.S. Pat. No. 7,078,518 yielded high levels of residual solvents (see Reference Examples 1A and 1B). On the other hand the 5-azacytidine of the present invention contains low levels of residual solvents. The inventors of the present invention also have found that when purification of 5-azacytidine was carried out overnight by crystallization from DMF at ambient temperature, the final product contained (after slurrying in acetone) 1780 ppm of DMF (Example 2). However, when purification of 5-azacytidine was carried out overnight by crystallization from DMF at a temperature of −20° C., the final product contained (after slurrying in acetone) only 165 ppm of DMF (Example 2A).

The 5-azacytidine obtained by the methods provided herein is stable under typical storage conditions for a solid, such as ambient temperatures (e.g., about 20° C. to about 30° C.) and relative humidities of up to about 60%. The term “stable” is used to refer to 5-azacytidine that retains at least about 85% of its initial amount under various storage conditions. In certain cases, the 5-azacytidine is stable after 1 month storage, after 2 months storage, after 3 months storage, after 4 months storage, after 5 months storage, or after 6 months storage. In some cases, the 5-azacytidine retains at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% of its initial amount

5-Azacytidine obtained by the methods provided herein can be used in pharmaceutical compositions for intravenous infusion or injection together with other acceptable additives and excipients, one non-limiting example of which is mannitol.

It has been found by the inventors of the present invention that a ready-to-use dosage of VIDAZA™ has a purity of the active pharmaceutical ingredient (API) (5-azacytidine) of only 98.7%. Furthermore, the sample analysis showed that significant quantities of 5-azacytidine degradation impurities were contained in the sample.

Thus, the present invention provides a method of analyzing a sample of 5-azacytidine to determine its purity and to identify and/or measure the impurities present in the sample. These analytical methods comprise the use of chromatography. The analyses of the samples are typically carried out using gas chromatography or liquid chromatography. Methods of liquid chromatography are, for example, Thin Layer Chromatography (TLC), High Pressure Liquid Chromatography (HPLC), and/or Liquid Chromatography/Mass spectrometry (LC-MS).

The method of analyzing a sample containing 5-azacytidine comprises:
separating 5-azacytidine and 5-azacytidine degradation products in the sample using a liquid chromatography system (LC), wherein the LC system is equipped with a suitable stationary phase and is capable of separating the 5-azacytidine and 5-azacytidine degradation products; and
identifying and/or detecting the presence and/or amount of the 5-azacytidine degradation products in the sample using mass spectrometry (MS).

The suitable stationary phase of the LC system, which facilitates separation of the constituents of the 5-azacytidine sample, typically is a Reverse Phase (RP) stationary phase column, which can be a C4, C8, C14, C18, phenyl, or polymeric packing, e.g., polyamide, polymethacrylate, polystyrene, and the like. In some specific embodiments, the LC is equipped with a C18 stationary phase.

The sample of 5-azacytidine can be any sample, including, for example, those used for injectable suspensions and commercially synthesized 5-azacytidine.

Thus, a sample of 5-azacytidine, which was withdrawn from the VIDAZA™ packaging for injectable suspension, was analyzed by using the method disclosed herein (see Example 7, below). Three impurities were identified, that is RGU, RGU-CHO and Compound VI

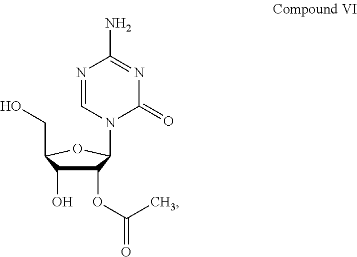

the results of which are summarized in Table 2.

TABLE 2

| | | | | | Molec- |
|---|---|---|---|---|---|
| | | m/z, | | Identified | ular |
| RRT* | Area % | M⁺¹ | MS major fragments | compound | weight |
| 0.35 | 0.12 | 235.1 | 150.1, 132.6, 86.1, 72.2 | RGU | 234.2 |
| 0.64 | 1.31 | 263.1 | 150.1, 132.6, 114.1, | RGU-CHO | 262.2 |
| | | | 87.9, 72.2 | | |
| 2.06 | 0.13 | 286.9 | 174.7, 113.1 | Compound VI | 286.2 |
| 1.00 | 98.45 | 244.9 | 133.0, 113.0, 85.9 | 5-azacytidine | 244.2 |

RRT = Relative Retention Time, where 1.00 is the retention time of 5-azacytidine

The results provided herein clearly demonstrate that the commercial 5-azacytidine sample, which was withdrawn from the VIDAZA™ packaging, has a purity of only 98.45%.

The present invention further provides a method of analyzing the structure of degradation products of a cytidine analogue, such as 5-azacytidine, to establish a degradation pathway of the cytidine analogue when exposed to degradation-inducing conditions.

The analysis of the impurity profiles of cytidine analogues, such as 5-azacytidine, formed under conditions of induced degradation can be performed using the methods disclosed herein, and, more specifically, using High Pressure Liquid Chromatography (HPLC), and/or Liquid Chromatography/Mass spectrometry (LC-MS), Fourier Transform Infra Red (FT-IR) spectroscopy, and a combination of methods thereof.

An induced degradation study on 5-azacytidine can be performed in solid state conditions, as well as in liquid state conditions. Solid state conditions include, but are not limited to, storage conditions, ambient conditions, elevated temperature conditions, UV light conditions, and accelerated conditions (e.g., high humidity and/or temperature). The liquid state conditions include but are not limited to, photolysis conditions, acidic conditions, basic conditions, and oxidative conditions.

Table 3 summarizes the various experimental conditions of induced degradation of 5-azacytidine. The diluent comprises a mixture of 30% 10 mM ammonium acetate and 70% THF.

TABLE 3

| | Degrada- | | | |
|---|---|---|---|---|
| | tion | | Experimental | |
| Entry | condition | State | conditions | Sample preparation |
| 1 | Storage | Solid | none | The sample was |
| | | | | withdrawn directly |
| | | | | from the package |
| 2 | Ambient | Solid | Exposure to visible | The sample of 5- |
| | | | light for 48 hours | azacytidine was |
| | | | at 25° C. | used as is |
| 3 | Elevated | Solid | Exposure to a tempera- | The sample of 5- |
| | tempera- | | ture of 105° C. for | azacytidine was |
| | ture | | 48 hours | used as is |
| 4 | UV light | Solid | Exposure to UV light | The sample of 5- |
| | | | for 48 hours at 25° C. | azacytidine was |
| | | | | used as is |
| 5 | Acceler- | Solid | Exposure to a tempera- | The sample of 5- |
| | ated | | ture of 40° C. and 75 | azacytidine was |
| | conditions | | relative humidity for | used as is |
| | | | 48 hours | |
| 6 | Photolysis | Liquid | Exposing a sample to | 50 mg of |
| | | | UV light for 48 hours | 5-azacytidine |
| | | | at 25° C. | was dissolved in |
| | | | | 50 ml of the diluent |
| 7 | Acid | Liquid | Exposing a sample at | 50 mg of |
| | hydrolysis | | 25° C. for one hour | 5-azacytidine |
| | | | | was dissolved in |
| | | | | 50 ml of 0.01M HCl |
| 8 | Basic | Liquid | Exposing a sample at | 50 mg of |
| | hydrolysis | | 25° C. for one hour | 5-azacytidine was |
| | | | | dissolved in 50 ml |
| | | | | of 0.01M NaOH |
| 9 | Oxidation | Liquid | Exposing a sample at | 50 mg of |
| | | | 25° C. for one hour | 5-azacytidine was |
| | | | | dissolved in 25 ml |
| | | | | of 10% hydrogen |
| | | | | peroxide solution |

Example 9 tests the induced degradation analysis of 5-azacytidine in solid state, wherein a slight change in color of the sample was observed when exposed to an elevated temperature. The FT-IR spectra did not show any significant changes. Furthermore, the HPLC analysis shows that the material is stable to heat and IJV light as long as it is in solid state, as detailed in Tables 7 and 8 respectively.

Example 10 tests the induced degradation analysis of 5-azacytidine in liquid state, wherein the HPLC analysis shows significant degradation, as detailed in Table 9.

Example 11 tests the solution stability of the 5-azacytidine in the experimental conditions of the HPLC method, as disclosed herein. The results, which are summarized in Table 10 below, indicate that 5-azacytidine is stable within the average time period needed to complete the HPLC method, while being dissolved in the HPLC diluent.

Example 12 tests the solution stability of the 5-azacytidine in water. The results, which are summarized in Table 11 below, indicate that 5-azacytidine is unstable in water over prolonged time periods.

The use of the terms “a” and “an” and “the” and similar referents in the context of describing the invention and, in the following claims, are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein. Variations of those preferred embodiments may become apparent to those of ordinary skill in the art upon reading the foregoing description. Accordingly, this invention includes all modifications and equivalents of the subject matter recited in the claims appended hereto.

### EXAMPLES

### Reference Example 1 (Prior Art Preparation)

This example demonstrates the preparation of 5-azacytidine according to prior art examples, e.g., Vorbrueggen et. al., J. Org. Chem. Vol. 39, No.25, 1974 and U.S. Pat. No. 7,038,038.

5-Azacytosine (200 g, 1.8 mol) was mixed with 1,1,1,3,3,3-hexamethyldisilazane (HMDS) (800 ml, 619.36 g, 3.837 mol) and ammonium sulfate (NH₄)₂SO₄ (5 g, 37.8 mmol). The resulting mixture was heated to reflux for a period of 5 hours. Then, the mixture was cooled to 60° C., and the excess HMDS was distilled off under reduced pressure. The residue was heated to 135° C. for 30 minutes, and the product was cooled to ambient temperature to afford bis(trimethylsilyl)-5-azacytosine (404 g, 1.58 mol). The 5-azacytosine was dissolved in dry 1,2-dichloroethane (125 ml), and 1,2,3,5-tetra-O-acetyl-β-D-ribofuranose (47 g, 0.1476 mol) was added. The reaction mixture was cooled to 5-10° C. and a solution of SnCl₄ (42.18 g, 0.162 mol) in 1,2-dichloroethane (25 ml) was added dropwise over 15 minutes. The resulting mixture was stirred for 2 hours, during which time the temperature was allowed to reach ambient temperature. Sodium bicarbonate (NaHCO₃) (70 g) was added under constant mixing and the reaction mixture was cooled to 15° C. Purified water (140 ml) was added drop wise and mixing was maintained for additional 20 minutes, then 1,2-dichloroethane was added and mixing was maintained for 10 additional minutes. The organic and aqueous phases were separated, and the organic phase was filtered through a layer of Celite, washed with 1,2-dichloroethane, and dried over sodium sulfate (Na₂SO₄).

The organic solvent was evaporated, and the residue was dissolved in methanol (120 ml), then heated to 60° C. to afford a clear solution. Charcoal (1.6 g) was added and the resulting mixture was stirred for 2 hours at ambient temperature. The charcoal was filtered off, and methanol/ammonia solution (200 ml of a 16% solution) was added to the filtrate and stirring was maintained for 20 hours at ambient temperature, during which time the reaction mixture solution gradually became viscous. Vacuum was applied to remove the excess ammonia, and the reaction mixture was cooled to 5° C. The resulting solid was filtered off, washed with methanol (3×30 ml) and dried to obtain crude 5-azacytidine (8 g, 21% yield) having purity of 98.7% (according to HPLC).

### Reference Example 1A (Prior Art Preparation)

This example demonstrates the purification of 5-azacytidine by crystallization according to Example 2 of U.S. Pat. No. 7,078,518.

5-azacytidine (5 g), having a purity of 98.7% and containing, inter alia, 0.14% by weight RGU-CHO and 0.09% by weight RGU, was dissolved in DMSO preheated to 90° C. (100 ml), and toluene preheated to 50° C. was added (900 ml) to the solution and mixed. The solution was cooled to ambient temperature overnight to form crystals. The resulting crystals were collected by filtration and air-dried to yield 5-azacytidine having a purity of 98.9% by weight, containing 0.33% by weight RGU-CHO. The sample contained 23.13% residual solvents, according to the TGA curve.

### Reference Example 1B (Prior Art Preparation)

This example demonstrates the purification of 5-azacytidine by crystallization according to Example 3 of U.S. Pat. No. 7,078,518.

5-azacytidine (5 g), having a purity of 98.7% and containing, inter alia, 0.14% by weight RGU-CHO and 0.09% by weight RGU, was dissolved in DMSO preheated to 90° C. (100 ml), and a co-solvent (methanol, toluene, or chloroform) preheated to 50° C. was added (900 ml) to the solution and mixed. The solution was cooled to −20° C. overnight to form crystals. The resulting crystals were collected by filtration and air-dried to yield 5-azacytidine having purity and residual solvents content as detailed in Table 4.

TABLE 4

| | | | RGU-CHO | Residual solvents |
|---|---|---|---|---|
| Entry | Solvent combination | Purity * | content * | content ** |
| 1 | DMSO/methanol | 99.4% | 0.06% | 13.77% |
| 2 | DMSO/toluene | 97.8% | 0.36% | 20.64% |
| 3 | DMSO/chloroform | 97.6% | 0.17% | 31.65% |

* According to HPLC.
** According to TGA curve

### Example 2

This example demonstrates the purification of 5-azacytidine by crystallization from N,N-dimethylformamide (DMF) at ambient temperature and slurrying in acetone.

In a 100 ml round flask, crude 5-azacytidine (0.5 g), having a purity of 98.7% and containing, inter alia, 0.14% by weight RGU-CHO and 0.09% by weight RGU, was mixed with DMF (10 ml), and the mixture was heated to 65° C. to afford complete dissolution. The solution was cooled to ambient temperature overnight to form crystals. The resulting crystals were collected by filtration, washed twice with DMF, and filtered to obtain a wet solid. The solid was slurried for four hours in dry acetone (20 ml), filtered, washed with acetone and dried under reduced pressure to yield 5-azacytidine having a purity of 99.6% by weight, containing 0.1% by weight RGU-CHO and 0.3% by weight of other impurities (as measured by HPLC). No traces of RGU were found in this sample. The sample contained 1780 ppm of DMF and 1340 ppm of acetone.

### Example 2A

This example demonstrates the purification of 5-azacytidine by crystallization from N,N-dimethylformamide (DMF) at a temperature of −20° C. and slurrying in acetone.

Crude 5-azacytidine (115 g), having a purity of 98.7% and containing, inter alia, 0.14% by weight RGU-CHO and 0.09% by weight RGU, was mixed with DMF (1725 ml), and the mixture was heated to 100° C. to afford complete dissolution. The solution was cooled under mixing to a temperature of −20° C. over a period of two hours and left at that temperature overnight to form crystals. The resulting crystals were collected by filtration, washed twice with acetone (2×50 ml) and filtered to obtain a wet solid. The solid was slurried at ambient temperature for 4 hours in acetone (3000 ml), filtered, washed twice with acetone (2×100 ml) and dried at a temperature of 80° C. under reduced pressure to yield 5-azacytidine having a purity of 99.95% by weight, containing 0.01% by weight RGU-CHO and 0.02% of RGU. The sample contained 165 ppm of DMF and 781 ppm of acetone.

### Example 3

This example demonstrates the purification of 5-azacytidine by crystallization from N,N-dimethylformamide (DMF).

In a 250 ml round flask, crude 5-azacytidine (5 g), having a purity of 98.7% by weight and containing, inter alia, 0.14% by weight RGU-CHO and 0.09% by weight RGU, was mixed with dry DMF (100 ml), and the mixture was heated to 100° C. to afford complete dissolution. The solution was cooled to ambient temperature, then to 5° C. overnight to form crystals. The resulting crystals were collected by filtration, washed twice with DMF, and dried at 80° C. under reduced pressure to yield 1.5 g of 5-azacytidine having a purity of 99.7% by weight and containing 0.27% by weight RGU-CHO and 0.03% by weight of other impurities (as measured by HPLC). No traces of RGU were found in this sample.

### Example 4

This example demonstrates the purification of 5-azacytidine by crystallization from N,N-dimethylacetamide (DMA).

In a 250 ml round flask crude 5-azacytidine (5 g), having a purity of 98.7% by weight and containing, inter alia, 0.14% by weight RGU-CHO and 0.09% by weight RGU, was mixed with dry DMF (50 ml), and the mixture was heated to 100° C. to afford complete dissolution. The solution was cooled to ambient temperature, then to 5° C. overnight to form crystals. The resulting crystals were collected by filtration, washed twice with DMF, and dried at 80° C. under reduced pressure to yield 5-azacytidine having a purity of 99.7% by weight and containing 0.22% by weight RGU-CHO and 0.08% by weight of other impurities (as measured by HPLC). No traces of RGU were found in this sample. The sample contained 2000 ppm of DMA

### Example 5

This example demonstrates the purification of 5-azacytidine by first crystallization from N,N-dimethylacetamide (DMA) and second crystallization from N,N-dimethylformamide (DMF).

In a 250 ml round flask crude 5-azacytidine (5 g), having a purity of 98.7% by weight and containing, inter alia, 0.14% by weight RGU-CHO and 0.09% by weight RGU, was mixed with dry DMA (50 ml), and the mixture was heated to 100° C. to afford complete dissolution. The solution was cooled to ambient temperature overnight to form crystals. The resulting crystals were collected by filtration and triturated twice with dry acetone. The wet material was mixed with dry DMF (50 ml), and the mixture was heated to 100° C. to afford complete dissolution. The solution was cooled to ambient temperature overnight to form crystals. The resulting crystals were collected by filtration, washed twice with DMF and dried at 80° C. under reduced pressure to yield 5-azacytidine having a purity of 99.7% by weight and containing 0.02% by weight RGU-CHO, 0.04% RGU by weight and 0.24% by weight of other impurities (as measured by HPLC).

### Example 6

This example demonstrates the purification of 5-azacytidine by crystallization from dimethylsufoxide (DMSO) and slurrying in acetone.

In a 100 ml round flask crude 5-azacytidine (1 g), having a purity of 98.7% by weight and containing, inter alia, 0.14% by weight RGU-CHO and 0.09% by weight RGU, was mixed with DMSO (2 ml), and the mixture was heated to 100° C. to afford complete dissolution. The solution was cooled to ambient temperature overnight to form crystals. The resulting crystals were collected by filtration, washed twice with DMSO, and filtered to obtain a wet solid. The solid was slurried for an hour with dry acetone (20 ml), filtered, and dried under reduced pressure to yield 5-azacytidine having a purity of 99.1% by weight and containing 0.26% by weight RGU-CHO and 0.64% by weight of other impurities (as measured by HPLC). No traces of RGU were found in this sample.

### Example 7

This example demonstrates the purification of 5-azacytidine by slurrying in acetone.

In a 100 round flask, crude 5-azacytidine (2g), having a purity of 98.7% by weight and containing, inter alia, 0.14% by weight RGU-CHO and 0.09% by weight RGU, was mixed with dry acetone (10 ml) at ambient temperature and left overnight to form a solid. The solid was collected by filtration, washed twice with acetone, and dried to yield 5-azacytidine having a purity of 99.5% by weight and containing 0.11% by weight RGU-CHO and 0.39% by weight of other impurities (as measured by HPLC), as depicted in Entry 5 of Table 3. No traces of RGU were found in this sample. The impurities profile which was obtained in several experiments which were carried out for purification of 5-azacytidine by slurrying in acetone, are further detailed in Table 5 marked as entries 1-4.

TABLE 5

| | | | | | | | | | | | Total other |
|---|---|---|---|---|---|---|---|---|---|---|---|
| | | | | | | | | | | | impurities |
| | Relative Retention Time (RRT) | | | | | | | | | | by % area |
| | 0.44 | 0.49 | 0.80 | 1.23 | 2.32 | 2.50 | 2.57 | 2.65 | 2.84 | 2.96 | (excluding |
| Entry | Area (%) | | | | | | | | | | RGU-CHO) |
| 1 | 0.01 | 0.04 | | 0.01 | 0.22 | | 0.03 | | | | 0.3 |
| 2 | | | | | | | | 0.02 | 0.01 | | 0.03 |
| 3 | 0.03 | 0.04 | 0.01 | | | | | | | | 0.08 |
| 4 | 0.04 | 0.10 | 0.08 | 0.02 | 0.06 | | 0.11 | 0.19 | 0.04 | | 0.64 |
| 5 | | 0.03 | 0.02 | | 0.07 | 0.08 | | | | 0.04 | 0.39 |

### Example 8

This example details HPLC method parameters for analyzing 5-azacytidine samples.

The HPLC measurements were performed using a system equipped with an Inertsil C18 column (ODS-2, 5 microns, 250×4.6 mm (ODS-167)). Other parameters of the system were as follows:
Detection: UV detector operated on 242 nm
Column temperature: 20° C.
Run time: 45 minutes
Injection volume: 10 μl
Flow rate: 1.0 ml/minute
Sample set temperature: 5° C.
Sample concentration: about 1.65 mg/ml
Diluent: Mixture of 30% 10 mM ammonium acetate and 70% THF

Analyses were performed using the following mobile phase
Mobile Phase (Eluent) A: 10 mM ammonium acetate
Mobile Phase (Eluent) B: 60% 10 mM ammonium acetate, 40% MeOH

The HPLC gradient is detailed in Table 6.

TABLE 6

| Time (minutes) | Eluent A % | Eluent B % |
|---|---|---|
| 0 | 95 | 5 |
| 10 | 95 | 5 |
| 20 | 30 | 70 |
| 45 | 30 | 70 |
| 45.1 | 95 | 5 |
| 52 | 95 | 5 |

### Example 9

This example details the preparation of samples for the induced degradation analysis in solid state.

Ambient conditions A 5-azacytidine sample (about 0.2 g) was spread uniformly in a Petri dish and exposed to visible light in the laboratory for 48 hours.
Elevated temperature A 5-azacytidine sample (about 0.2 g) was spread uniformly in a Petri dish and exposed to 105° C. for 48 hours.
UV light (Photolysis) A 5-azacytidine sample (about 0.2 g) was spread uniformly in a Petri dish as a thin layer and was covered with a transparent glass Petri dish lid. The sample was placed in a UV chamber and exposed to UV light for 48 hours.
Accelerated conditions [40±2° C./75±5% Relative Humidity (RH)]. A 5-azacytidine sample (about 0.2 g) was spread uniformly in a Petri dish and exposed to 40±2° C./75±5% relative humidity for 48 hours.

At the end of the stipulated time period, the physical descriptions of each sample were noted down. Identification tests were performed by FT-IR, and purity checks were performed by HPLC analysis. The protected sample, as defined herein, is the reference storage material used for carrying out the experiments detailed in Tables 7 and 8.

The results of induced degradation study of 5-azacytidine in solid state by observation as well as FT-IR tests is summarized in Table 7.

TABLE 7

| | Period of | | |
|---|---|---|---|
| Degradation | exposure | Observation | |
| conditions | (hours) | Description | IR spectrum |
| Protected | — | White to off | — |
| sample | | white powder | |
| Ambient | 48 | White to off | Comparable with protected |
| conditions | | white powder | sample IR spectrum |
| Elevated | 48 | Off white to cream | Comparable with protected |
| temperature | | color powder | sample IR spectrum |
| UV light | 48 | White to off | Comparable with protected |
| | | white powder | sample IR spectrum |
| Accelerated | 48 | White to off | Comparable with protected |
| conditions | | white powder | sample IR spectrum |

Table 8 below details the results obtained by HPLC measurements for solid state degradation.

TABLE 8

| | Test results (by HPLC) | | | | |
|---|---|---|---|---|---|
| | Relative Retention Time (RRT) * | | | | Total |
| | 0.63 | 1.00 | 2.01 | 2.05 | impuri- |
| Degradation conditions | Area (%) | | | | ties (%) |
| Protected sample (storage) | 1.55 | 96.72 | 1.37 | 0.24 | 3.28 |
| Exposure to ambient | 1.52 | 96.75 | 1.36 | 0.27 | 3.25 |
| conditions | | | | | |
| Exposure to elevated | 2.00 | 96.61 | 1.01 | 0.17 | 3.39 |
| temperature (105° C.) | | | | | |
| Exposure to UV light | 1.66 | 96.64 | 1.35 | 0.24 | 3.36 |
| Accelerated conditions | 1.90 | 97.04 | 0.80 | 0.14 | 2.96 |
| (40 ± 2° C., 75 ± 5% RH) | | | | | |

* RRT of 5-azacytidine (set at 1.00). RH = Relative Humidity. The differences in the results are within the experimental error.

### Example 10

This example details the preparation of samples for the induced degradation analysis of liquid conditions.

Acidic hydrolysis—blank preparation: Hydrochloric acid (5 ml, 0.01M HCl) was diluted to 10 ml with the diluent. Acidic hydrolysis—Preparation of sample solution: A 5-azacytidine sample (50 mg) was dissolved in 0.01M HCl (25 ml) and mixed at room temperature for about 1 hour. An aliquot (5 ml) was diluted to 10 ml with the diluent. The blank preparation and sample preparation were injected to the HPLC system by using the chromatographic conditions as mentioned in example 8.
Basic hydrolysis—blank preparation: Sodium hydroxide (5 ml, 0.01M NaOH) was diluted to 10 ml with the diluent. Basic hydrolysis—preparation of sample solution: A 5-azacytidine sample (50 mg) was dissolved in 0.01M NaOH (25 ml) and mixed at room temperature for about 1 hour. An aliquot (5 ml) was diluted to 10 ml with diluent. The blank preparation and sample preparation were injected to the HPLC system using the chromatographic conditions as detailed in example 8.
Oxidation—blank preparation: Hydrogen peroxide (5 ml, 10% solution) was poured into a clean and dry 10 ml volumetric flask and filled up to the mark with the diluent.
Oxidation—preparation of sample solution: A 5-azacytidine sample (50 mg) was dissolved in 10% hydrogen peroxide solution (25 ml) and mixed at room temperature for about 1 hour. An aliquot (5 ml) was diluted to 10 ml with the diluent. The blank and sample preparations were injected to the HPLC system using the chromatographic conditions as detailed in example 8.
Photolysis—blank preparation: The diluent (50 ml) was mixed under UV light for 48 hours. Photolysis—preparation of sample solution: A 5-azacytidine sample (50 mg) was dissolved in the diluent (50 ml) and the solution was exposed to UV light under mixing for 48 hours. The blank preparation and sample preparation were injected to the HPLC system using the chromatographic conditions as mentioned in example 8.

Table 9 below details the results obtained for liquid state degradation

TABLE 9

| | Test results (by HPLC) | | | | | | | | | |
|---|---|---|---|---|---|---|---|---|---|---|
| | Relative Retention Time (RRT)* | | | | | | | | | Total |
| Degradation | 0.33 | 0.39 | 0.42 | 0.63 | 1.00 | 1.18 | 1.65 | 2.01 | 2.05 | impurities |
| conditions | Area (%) | | | | | | | | | (%) |
| Storage | — | — | — | 1.55 | 96.72 | — | — | 1.37 | 0.24 | 3.28 |
| Acidic | 0.17 | — | — | 21.1 | 71.96 | 5.35 | 0.14 | 1.10 | — | 28.04 |
| Basic | 89.92 | — | 3.97 | — | 3.38 | — | — | — | — | 96.62 |
| Oxidation | 0.84 | — | 0.06 | 0.23 | 97.18 | — | — | 1.62 | — | 2.82 |
| Photolysis | 0.85 | 0.11 | 0.20 | 35.24 | 61.64 | 0.46 | 0.39 | 0.86 | 0.17 | 38.36 |

### Example 11

This example details the solution stability of the 5-azacytidine in the experimental conditions of the HPLC method.

A sample of 5-azacytidine in the diluent (about 1.65 mg/ml) was withdrawn from the flask (which was kept at the HPLC conditions as detailed in example 7) on every consecutive hour and injected to the HPLC system. The results, which are summarized in Table 10, demonstrate the stability of 5-azacytidine in prolonged dilution in the HPLC diluent.

TABLE 10

| | Relative Retention Time (RRT) * | | | | Total |
|---|---|---|---|---|---|
| Time | 0.63 | 1.00 | 2.01 | 2.05 | impurities |
| (Hours) | Area (%) | | | | by % area |
| 0 | 1.55 | 96.72 | 1.37 | 0.24 | 3.28 |
| 4 | 2.04 | 96.21 | 1.41 | 0.24 | 3.79 |
| 5 | 2.16 | 96.11 | 1.37 | 0.24 | 3.89 |
| 6 | 2.30 | 96.00 | 1.36 | 0.24 | 4.00 |
| 7 | 2.43 | 95.85 | 1.37 | 0.23 | 4.15 |
| 8 | 2.55 | 95.74 | 1.37 | 0.24 | 4.26 |
| 9 | 2.66 | 95.63 | 1.37 | 0.24 | 4.37 |
| 10 | 2.77 | 95.50 | 1.37 | 0.24 | 4.50 |
| 11 | 2.87 | 95.40 | 1.37 | 0.24 | 4.60 |

* RRT of 5-azacytidine

### Example 12

This example details the solution stability of the 5-azacytidine in water.

A sample of 5-azacytidine was dissolved in water in a flask to form a solution having concentration of about 1.65 mg/ml. Samples were withdrawn from the flask every consecutive hour and injected to the HPLC system. The results, which are summarized in Table 11, demonstrate the instability of 5-azacytidine in prolonged dilution in water.

TABLE 11

| | Relative Retention Time (RRT) * | | | | Total |
|---|---|---|---|---|---|
| Time | 0.63 | 1.00 | 1.70 | 2.01 | impurities |
| (Hours) | Area (%) | | | | by % area |
| 0 | 0.64 | 97.47 | — | 1.59 | 2.53 |
| 1 | 2.16 | 95.97 | — | 1.57 | 4.03 |
| 4 | 6.86 | 91.38 | — | 1.46 | 8.62 |
| 8 | 14.68 | 83.61 | 0.23 | 1.36 | 16.39 |
| 12 | 21.28 | 77.01 | 0.35 | 1.25 | 22.99 |

* RRT of 5-Azacytidine

What is claimed:

1. A method of purifying 5-azacytidine comprising:
(a) heating a solution of crude 5-azacytidine to at least 45° C.;
(b) allowing the solution of step (a) to cool to precipitate crystals of purified 5-azacytidine from the solution;
(c) optionally isolating, washing, and drying the crystals of step (b); and
(d) optionally slurrying the crystals of step (c) in a solvent, and filtering and drying the filtered crystals,
wherein the crystals of 5-azacytidine of step (b), (c), or (d) have a purity of at least 99.0% by weight of 5-azacytidine and contain up to 0.2% by weight of any individual degradation product of 5-azacytidine.

2. The method of claim 1, wherein the crystals of 5-azacytidine of step (b), (c), or (d) contain less than 0.1% by weight of any individual degradation product of 5-azacytidine.

3. The method of claim 1, wherein the solution of crude 5-azacytidine comprises a solvent selected from the group consisting of N,N-dimethylformamide, N,N-dimethylacetamide, ethylene glycol, N-methyl-2-pyrrolidone, dimethylsulfoxide, and mixtures thereof.

4. The method of claim 3, wherein the solution of crude 5-azacytidine comprises N,N-dimethylformamide, N,N-dimethylacetamide, or a mixture thereof.

5. The method of claim 1, wherein the solvent of step (d) comprises acetone, methyl ethyl ketone, methyl isobutyl ketone, ethyl acetate, n-propyl acetate, isoproyl acetate, n-butyl acetate, isobutyl acetate, ethanol, or a mixture thereof.

6. The method of claim 1, wherein the ratio 5-azacytidine: solvent of the crude 5-azacytidine to the solvent of step (a) is about 1 g 5-azacytidine per at least 2 ml solvent.

7. The method of claim 6, wherein the 5-azacytidine: solvent ratio is 1 g 5-azacytidine per 10 to 20 ml solvent.

8. The method of claim 1, wherein the 5-azacytidine has a purity of at least 99.0% by weight.

9. The method of claim 8, wherein the 5-azacytidine has a purity at least 99.6% by weight.

10. 5-azacytidine having less than 0.2% by weight of N-(formylamidino)-N′-β-D-ribofuranosylurea.

11. The 5-azacytidine of claim 10 having less than 0.1% by weight of N-(formylamidino)-N′-β-D-ribofuranosylurea.

12. 5-azacytidine having less than 0.1% by weight of 1-β-D-ribofuranosyl-3-guanylurea.

13. 5-azacytidine containing less than 200 ppm DMF and/or less than 1000 ppm acetone as residual solvents.

14. A pharmaceutical composition comprising the 5-azacytidine of claim 8 and a pharmaceutically acceptable excipient.

15. The pharmaceutical composition of claim 14, further comprising mannitol.

16. The method of claim 1, wherein the crystals of step (b), (c), or (d) are stable under storage conditions for at least 3 months.
